(19) United States
(12) Patent Application Publication
(10) Pub. No.: US 2013/0084615 A1
(43) Pub. Date: Apr. 4, 2013

(54) Method for Producing Ethanol and Yeast Protein Feed from Whey Permeate

(71) Applicant: Joseph Van Groll, Marshfield, WI (US)

(72) Inventor: Joseph Van Groll, Marshfield, WI (US)

(21) Appl. No.: 13/250,774

(22) Filed: Sep. 30, 2011

(51) Int. Cl. C12P 7/06

(52) U.S. Cl. 435/161

(57) ABSTRACT

A method for production of ethanol and yeast protein provides a lactose extraction and conversion process resulting in ≧8.5% V/V of alcohol after fermentation with up to ≧95% lactose conversion efficiency. Fermentation time is 24 hours and monitoring and adjusting of pH throughout the process in not required. In addition to ethanol, yeast protein feed is extracted after fermentation and provides up to 28% protein content as dry matter.

### CROSS-REFERENCE TO RELATED APPLICATIONS

This parent application is related to U.S. patent application Ser. No. 13/222,755, filed Aug. 31, 2011 and entitled “Ethanol and Protein Feed Plant Production System Using Whey Permeate,” which is hereby incorporated by reference in its entirety.

### FIELD OF INVENTION

The present invention relates to the field of ethanol production, and more specifically the production of ethanol from whey permeate.

### BRIEF DESCRIPTION OF THE DRAWINGS

FIG. 1 is a flow chart illustrating an exemplary embodiment of a method for producing ethanol and yeast protein feed from whey permeate,

### GLOSSARY

As used herein, the term “fermentation” means the conversion of sugar to carbon dioxide and alcohol by a microbe, e.g., the conversion of lactose to ethanol by yeast.

As used herein, the term “inoculation” refers to the introduction of a microorganism (e.g. strain of yeast) onto a growth medium (e.g. whey permeate).

As used herein, the term “propagation” refers to exponential growth of yeast cultures on whey permeate.

As used herein, the term “seeding” refers to the initiation of growth of yeast cultures on whey permeate.

As used herein, the term “whey permeate” refers to a modified dairy product that is obtained by removing protein from whey.

### BACKGROUND

Industrial processes for producing ethanol from whey permeate, a common waste byproduct of cheese production, is desirable because whey permeate is a pollutant, and ethanol can be used for biofuel. Land disposal of surplus whey can endanger the physical and chemical structures of soil, decrease crop yields, and cause serious water pollution. Disposal of surplus whey can be a significant expensive for businesses as municipalities typically charge per gallon of sewage. In addition, surcharges are often incurred for each pound of biochemical oxygen demand, a water pollutant contained in whey permeate. Other water pollutants in whey permeate include chemical oxygen demand, and some municipalities add surcharges for this waste as well. Creating products from whey reduces whey disposal costs for cheese producers, enabling their businesses to become more profitable. Thus, it is desirable to find inexpensive and environmentally-friendly alternatives for cheese producers to create commercially-desirable products from whey.

An estimated 90.5 billion pounds of whey was generated as a waste byproduct of cheese production in 2006, and they contained 4.4 billion pounds of lactose. An estimated total of 1.9 billion pounds of lactose were used to produce useful whey products. Therefore, about 2.5 billion pounds of surplus lactose were not used to produce whey products and remained as surplus. This vast amount of surplus lactose could be fermented to produce an estimated 203 million gallons of ethanol, assuming a theoretical ethanol conversion efficiency of 100%.

Current methods for the fermentation of whey typically require multiple batches, in some cases up to eight batches. Fermentation of each batch requires more than 24 hours, This means that the total fermentation process requires more than eight days to produce biofuel.

Current methods also typically require multiple steps which increase production cost and time, e.g., monitoring and controlling pH of the whey permeate throughout the process.

No fermentation method known in the art ferments ethanol from whey permeate in 24 hours in very few steps, which is desirable because whey permeate can easily spoil or change to a temperature that is not useful for fermentation. No process to produce ethanol from whey permeate known in the art allows whey permeate to be unloaded from trucks into holding tanks, which is desirable because it permits fermentation at facilities that are located away from dairy plants that produce whey permeate as a waste byproduct.

### SUMMARY OF THE INVENTION

The present invention is a method for producing ethanol and yeast protein feed by fermenting whey permeate, a dairy waste product. Whey permeate is first pumped into the inoculation tank, and air feed tubes add various chemicals and antibiotics. The purpose of the inoculation tank is to introduce a strain of yeast which can ferment whey permeate. The contents of the inoculation tank are heated and transferred to a seeding tank, which initiates the growth of yeast cultures onto whey permeate. Whey permeate and the contents of the inoculation tank are pumped into the seeding tank, and various chemicals and antibiotics are added by air feed tubes. The contents of the seeding tank are heated before being transferred to the propagation tank, which exponentially grows yeast cultures on the whey permeate.

Whey permeate and the contents of the seeding tank are pumped into the propagation tank, and various chemicals and antibiotics are added by air feed tubes. The contents of the propagation tank are heated before being transferred to the fermentation tank. Whey permeate and the contents of the propagation tank are pumped into the fermentation tank, and various chemicals and antibiotics are added by air feed tubes. The contents of the fermentation tank are heated to ferment whey permeate into ethanol and a yeast protein feed.

The fermentation process for producing ethanol and yeast protein feed from whey permeate lasts 24 hours. Ethanol is useful as a biofuel, and the yeast protein feed can be used for ruminant animals. The method to produce ethanol from whey permeate can pump the ethanol from the fermentation tank to holding tanks for transport by motor vehicles,

### DETAILED DESCRIPTION OF INVENTION

For the purpose of promoting an understanding of the present invention, references are made in the text to exemplary embodiments of a method for producing ethanol and yeast protein feed from whey permeate, only some of which are described herein. It should be understood that no limitations on the scope of the invention are intended by describing these exemplary embodiments. One of ordinary skill in the art will readily appreciate that alternate but functionally equivalent processes for producing ethanol from whey permeate at an industrial scale may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

Moreover, the terms “substantially” or “approximately” as used herein may be applied to modify any quantitative representation that could permissibly vary without resulting in a change in the basic function to which it is related.

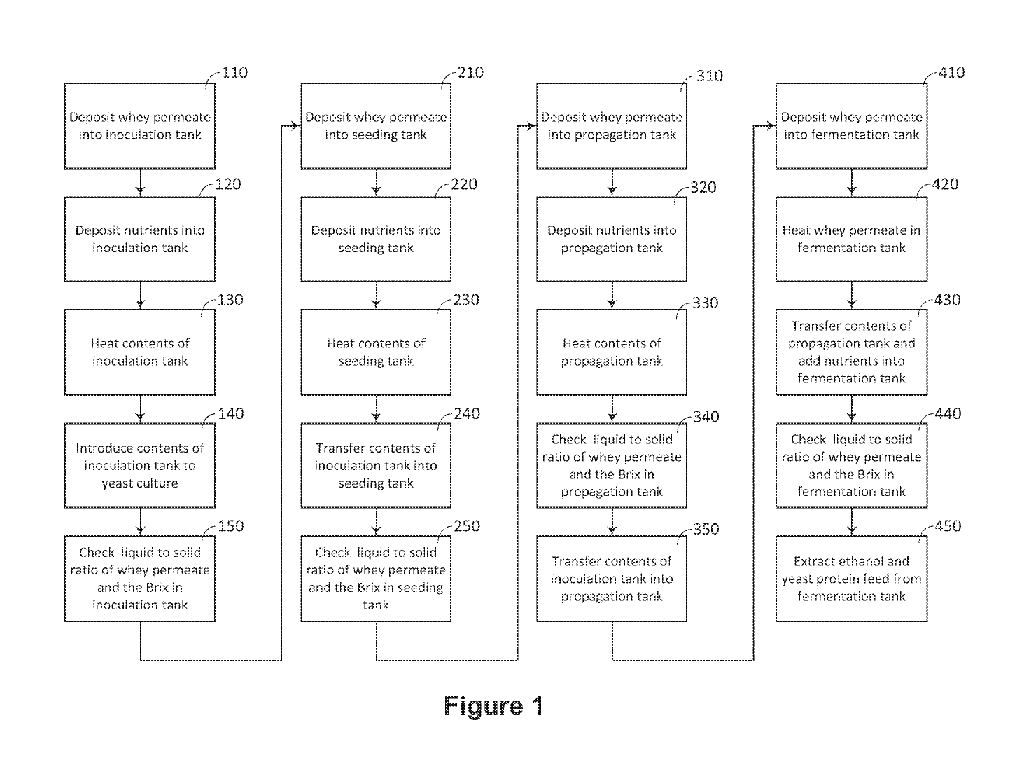

FIG. 1 illustrates an exemplary embodiment of method for producing ethanol and yeast protein feed from whey permeate. In the exemplary embodiment shown, fermentation time is 24 hours and monitoring and adjusting the pH throughout the process is not required.

The method illustrated in FIG. 1 comprises a first Step 110 of depositing whey permeate into an inoculation tank. The method comprises a second Step 120 of depositing nutrients into the inoculation tank. The method comprises a third Step 130 of heating contents of the inoculation tank. The method comprises a fourth Step 140 of depositing a yeast culture into the inoculation tank. The method comprises a fifth Step 150 of checking the liquid to solid ratio and the Brix level in the inoculation tank.

The method comprises a sixth Step 210 of depositing whey permeate into a seeding tank. The method comprises a seventh Step 220 of depositing nutrients into the seeding tank. The method comprises an eighth Step 230 of heating the contents of the seeding tank. The method comprises a ninth Step 240 of transferring the contents of the inoculation tank into the seeding tank. The method comprises a tenth Step 250 of checking the liquid to solid ratio and the Brix level in the seeding tank.

The method comprises an eleventh Step 310 of depositing whey permeate into a propagation tank, The method comprises a twelfth Step 320 of depositing nutrients into the propagation tank. The method comprises a thirteenth Step 330 of heating contents of the propagation tank. The method comprises a fourteenth Step 340 of transferring the contents of the seeding tank into the propagation tank. The method comprises an fifteenth Step 350 of checking the liquid to solid ratio and the Brix level in the propagation tank.

The method comprises a sixteenth Step 410 of depositing whey permeate into a fermentation tank. The method comprises a seventeenth Step 420 of heating the contents of the fermentation tank. The method comprises an eighteenth Step 430 of transferring the contents of the propagation tank and depositing nutrients into the fermentation tank. The method comprises a nineteenth Step 440 of checking the liquid to solid ratio and the Brix level in the fermentation tank. The method comprises a twentieth Step 450 of extracting ethanol and yeast protein feed from the fermentation tank.

In Step 110 of the embodiment shown in FIG. 1, an inoculation tank receives whey permeate from an unloading pump. In an exemplary embodiment, the inoculation tank has a capacity of 60 gallons. The size and the configuration of the inoculation tank may be adjusted to accommodate production size and volume needs.

In the exemplary embodiment, 55 gallons of whey permeate, 11-12% of which is solid, is deposited in said inoculation tank.

In some exemplary embodiments, inoculation tank may receive whey permeate from a truck, or whey permeate may be placed in a temporary storage container to be added to an inoculation tank later.

In Step 120, nutrient tubes deposit nutrients into the inoculation tank containing whey permeate. In the exemplary method described, nitrogen, phosphorus and corn steep liquor are all deposited by individual nutrient tubes into the inoculation tank. In some exemplary embodiments, nitrogen may be deposited in the form of urea ammonium nitrate and phosphorus may be deposited in the form of liquid ammonium phosphate. Other nutrients which may be deposited at this time include antibiotics and hydrogen peroxide.

In the exemplary embodiment described, one quart of liquid ammonium phosphate, one quart of corn steep liquor, and one ounce of antibiotic or antimicrobial solution, such as ALL-Pen antibiotic known in the art, are deposited into inoculation tank. Ammonium phosphate provides a source of phosphorous nutrient for yeast growth. Corn steep liquor provides a source of vitamins and growth factors for yeast growth.

One or more antibacterial or antimicrobial agents are added to inhibit bacterial growth or contamination of the yeast culture. In this exemplary embodiment, one ounce of antibiotic feed is deposited into said inoculation tank. Antibiotics inhibit bacterial growth and contamination of the yeast culture.

The amount of liquid ammonia phosphate and corn steep liquor, as well as the amount and type or combination of antibiotics used, may be adjusted to accommodate the amount of whey permeate introduced or other specific production parameters or needs.

The contents of inoculation tank are mixed, e.g., with an agitator, to form an inoculation mixture from the whey permeate, liquid ammonium phosphate, corn steep liquor, and antibiotic.

In Step 130, the contents of the inoculation tank are heated to the appropriate temperature with a heating element. A heating element may include, but is not limited to, a heating pump, an immersion heater, and a boner. In one exemplary embodiment, the contents of inoculation tank are heated to 85 degrees Fahrenheit. In further exemplary embodiments, the inoculation tank may be heated to a higher or lower temperature. To assist in regulating temperature, the inoculation tank may be jacketed. The jacket maintains the contents of said inoculation tank at 85 degrees Fahrenheit.

In Step 140, the contents of the inoculation tank are exposed to a yeast culture (e.g. Kluyveromyces marxianus). In the presence of the proper nutrients, the yeast culture converts the lactose sugar to carbon dioxide and ethyl alcohol, i.e., ethanol. In the exemplary embodiment, half a gallon of yeast culture Kluyveromyces marxianus is used. Further exemplary embodiments may use different amounts of Kluyveromyces marxianus or different strains of yeast culture.

In Step 150, the whey permeate in the inoculation tank is checked for its liquid to solid ratio and its Brix level. A hydrometer (or similar instrument capable of measuring the ratio between the density of the measured liquid to the density of water) is used to check the liquid to solid ratio of the whey permeate and the Brix level. In some exemplary embodiments, a sugar hydrometer, which is a Brix measurement device, may be used.

In the exemplary embodiment described, the desired ratio is reached in approximately 8 hours. A timer may be set for eight hours and the ratio tested at that time and reset for additional time if the desired ratio has not been achieved, Other embodiments may set the timer for a different time correlating to the size of inoculation tank or amount of inoculation mixture.

In Step 210, a seeding tank receives whey permeate from an unloading pump. The purpose of a seeding tank is to initiate the growth of yeast cultures onto whey permeate. In the exemplary embodiment shown in FIG. 1, 500 gallons of whey permeate, 11 to 12% of which is solid, is deposited into said seeding tank. Still further exemplary embodiments may use different amounts of whey permeate or use whey permeate with a different liquid to solid ratio. In this exemplary embodiment, the seeding tank has a capacity of 600 gallons. The size of the seeding tank may be adjusted to accommodate smaller or larger production needs.

In Step 220, air feed tubes deposit nutrients into the seeding tank, including, but not limited to, liquid ammonium phosphate, UAN, corn steep liquor and antibiotics. One or more sources of nutrients are added to the seeding tank. In an exemplary embodiment, one gallon of liquid ammonium phosphate, one quarter gallon of UAN, and one half gallon of corn steep liquor are deposited into said seeding tank.

One or more antibacterial or an antimicrobial agents are added to inhibit bacterial growth or contamination of the yeast culture. In the exemplary embodiment described, one ounce of antibiotics is deposited into said inoculation tank.

The amount of liquid ammonia phosphate, corn steep liquor, and UAN, as well as the amount and type or combination of antibacterial and antimicrobial agents used may be adjusted to accommodate the amount of whey permeate that is used.

The contents of seeding tank are mixed, e.g., with an agitator, to form a seeding mixture from the inoculation mixture, whey permeate, liquid ammonium phosphate, corn steep liquor, UAN, antibiotic, and hydrogen peroxide.

In Step 230, the contents of the seeding tank are heated to the appropriate temperature by a heating element. A heating element may include but is not limited to a heating pump, an immersion heater, and a boiler. In one exemplary embodiment, the contents of seeding tank are heated to 88 degrees Fahrenheit. The seeding mixture is incubated for a desired time at a desired temperature. To assist in regulating temperature, the seeding tank may be placed within a jacketed tank chamber. The seeding tank jacket maintains the contents of the seeding tank at a desired temperature. In further exemplary embodiments, the seeding tank may be heated to a higher or lower temperature based on various climate variables such as room temperature and altitude.

In Step 240, the contents of the inoculation tank are transferred to the seeding tank. In Step 250, the content of the seeding tank is checked for its liquid to solid ratio and its Brix level. Desirably, a ratio of at least three degrees Brix is achieved prior to transfer of the seeding tank contents into the propagation tank. In the exemplary embodiment described, the desired ratio is reached in approximately 8 hours.

In Step 310, a propagation tank receives whey permeate from an unloading pump. The purpose of a propagation tank is to exponentially grow yeast cultures on the whey permeate. In the exemplary embodiment, 5,000 gallons of whey permeate, 12 to 13% of which is solid, is deposited into said propagation tank. Still further exemplary embodiments may use different amounts of whey permeate or use whey permeate with a different liquid to solid ratio. In one exemplary embodiment, the propagation tank has a capacity of 6,000 gallons. The size of the propagation tank may be adjusted to accommodate smaller or larger production needs.

In Step 320, nutrients, such as liquid ammonium phosphate, corn steep liquor, antibiotics, antimicrobials and hydrogen peroxide are deposited into the propagation tank. One or more sources of nutrients are added to the propagation tank. This is preferably done through air feed tubes, In an exemplary embodiment, one gallon of liquid ammonium phosphate and three gallons of UAN are deposited into said propagation tank. In some exemplary embodiments, corn steep liquor may be added to the propagation tank; however, corn steep liquor may be omitted, as the mixture may already have the nutrients which it provides. One or more antibacterial or antimicrobial agents are added to inhibit bacterial growth or contamination of the yeast culture. In an exemplary embodiment, two ounces of antibiotics and one quarter gallon of hydrogen peroxide are deposited into the propagation tank. This is preferably done through air feed tubes.

The amount of liquid ammonia phosphate, corn steep liquor, and UAN, as well as the amount and type or combination of antibacterial and antimicrobial agents used may be adjusted to accommodate the amount of whey permeate introduced or other specific production parameters or needs.

The contents of propagation tank are mixed with an agitator to form a propagation mixture from the whey permeate, liquid ammonium phosphate, corn steep liquor (if added), UAN, antibiotic, and hydrogen peroxide.

The amount and ratio of the contents of the propagation mixture y be adjusted to accommodate materials availability or specific production needs. For example, some exemplary embodiments may use different amounts of whey permeate or use whey permeate with a different liquid to solid ratio. Further exemplary embodiments may use different amounts of liquid ammonium phosphate, UAN, corn steep liquor, antibiotic, or hydrogen peroxide. Still further exemplary embodiments may use a different antibiotic feed or even a combination of antibiotic feeds.

In Step 330, the contents of the propagation tank are heated to the appropriate temperature by a heating element. A heating element may include but is not limited to a heating pump, an immersion heater and a boiler. In one exemplary embodiment, the contents of propagation tank are heated to a temperature between 90 and 91 degrees Fahrenheit. In further exemplary embodiments, the propagation tank may be heated to a higher or lower temperature. To assist in regulating temperature, the propagation tank may be placed within a jacketed tank chamber. The jacketed tank chamber heats and maintains the contents of the propagation tank at a desired temperature.

In Step 340, the contents of said seeding tank are transferred to the propagation tank by a transfer pump.

In Step 350, the whey permeate in the propagation tank is checked for its liquid to solid ratio and its Brix level. A sugar hydrometer is used to check the liquid to solid ratio of the whey permeate and the Brix level. Desirably, the contents of the propagation tank are at least three degrees Brix prior to transfer of the propagation tank contents to the fermentation tank. In the exemplary embodiment described, the desired liquid to solid ratio is reached in approximately 8 to 10 hours.

In Step 410, a fermentation tank receives whey permeate from an unloading pump. The purpose of a fermentation tank is to create ethanol and yeast protein feed by breaking down the carbohydrates of the whey permeate by the enzymes from the yeast cultures that were introduced in said inoculation tank and grown in the other previous tanks.

In one exemplary embodiment, the fermentation tank has a capacity of 35,000 gallons. The size of the fermentation tank may be adjusted to accommodate smaller or larger production needs. In the exemplary embodiment, 30,000 gallons of whey permeate, 16 to 19% of which is solid, is deposited into the fermentation tank. Still further exemplary embodiments may use different amounts of whey permeate depending on desired production levels.

In Step 420, the whey permeate in the fermentation tank is heated to the appropriate temperature. In one exemplary embodiment, the contents of fermentation tank are heated to a temperature between 95 and 98 degrees Fahrenheit. In further exemplary embodiments, the fermentation tank may be heated to a higher or lower temperature. The fermentation mixture is incubated for a desired time at a desired temperature. To assist in regulating temperature, the fermentation tank may be placed within a jacketed tank chamber. The tank jacket maintains the temperature of the contents of the fermentation tank between 95 and 98 degrees Fahrenheit or other desired temperature range.

In Step 430, UAN, the contents of the propagation tank, and antibiotics/antimicrobials are deposited into the fermentation tank. In the exemplary embodiment, 3,000 gallons of contents from the propagation tank are transferred to the fermentation tank. The amount transferred may be adjusted to accommodate different production needs.

Also in Step 430, one or more sources of nutrients are added to the fermentation tank, e.g., by air tube. In this exemplary embodiment, 10 gallons of UAN is deposited into said fermentation tank.

One or more antibacterial and/or antimicrobial agents are added to inhibit bacterial growth or contamination of the yeast culture. In this exemplary embodiment, five ounces of antibiotic feed are deposited into the fermentation tank.

The contents of the fermentation tank are mixed with an agitator to form a fermentation mixture from the propagation mixture, whey permeate, UAN, and antibiotics.

The amount and ratio of the contents of the fermentation mixture may be adjusted to accommodate materials availability or specific production needs. For example, exemplary embodiments may use different amounts of whey permeate or use whey permeate with a different liquid to solid ratio. Further exemplary embodiments may use different amounts of UAN or antibiotic feed. Still further exemplary embodiments may use a different antibiotic feed or even a combination of antibiotic feeds.

In Step 440, the liquid to solid ratio and Brix level of the whey permeate within the fermentation tank is checked. A sugar hydrometer is used to check the solid to liquid ratio at regular intervals. In the exemplary embodiment described, the desired ratio is reached in approximately 20 to 24 hours.

In Step 450, the ethanol and yeast protein feed is extracted from the fermentation tank.

Processing of ethanol and yeast protein feed by extraction may be undertaken at levels of ≧8.5% V/V of alcohol with up to ≧95% lactose conversion efficiency. Yeast protein feed is extracted after fermentation and provides up to 28% protein content as dry matter. Fermentation is ended when all lactose is converted. The contents will be three degrees Brix.

What is claimed is:

1. A method for producing ethanol and protein yeast feed comprising the steps of:
inoculating a first quantity of whey permeate with a first quantity of yeast and a first quantity of at least one nutrient source to create an inoculation mixture;
incubating said inoculation mixture at a temperature between 85 and 95 degrees Fahrenheit;
seeding said inoculation mixture by adding a second quantity of whey permeate and a second quantity of a nutrient source to create a seeding mixture;
incubating said seeding mixture at a temperature between 85 and 95 degrees Fahrenheit;
propagating said seeding mixture by adding a third quantity of whey permeate and a third quantity of a nutrient source to form a propagation mixture;
combining said propagation mixture with a fourth quantity of whey permeate and a fourth quantity of a nutrient source to form a propagating mixture;
fermenting said propagating mixture; and
extracting a quantity of ethanol and a quantity of yeast.

2. The method of claim 1 wherein the ratio of said first quantity of yeast added to said first quantity of whey permeate is approximately ½ gallon per 55 gallons.

3. The method of claim 1 wherein a first quantity of an antibacterial agent is added with said yeast during said inoculation.

4. The method of claim 3 wherein the ratio of said first quantity of an antibacterial agent added to said first quantity of whey permeate during said inoculation is approximately 1 ounce per 55 gallons.

5. The method of claim 3 wherein said first quantity of antibacterial agent is selected from the group consisting of antibacterial alternatives approved for safe human and animal consumption, chlorine dioxide, hydrogen peroxide, phosphorus, hydrochloric acid, tetracycline, and synthetic antimicrobials effective against pathogenic bacteria resistant to current antibacterials.

6. The method of claim 1 wherein said inoculation mixture is created in an inoculation tank.

7. The method of claim 1 which further includes the step of determining a first quantity of said yeast to be added, wherein the quantity of said yeast is between gallon and 1 gallon.

8. The method of claim 1 which further includes the step of selecting said first quantity of nutrient source, wherein said nutrient source is selected from the group consisting of liquid ammonium phosphate, urea ammonium nitrate, corn steep liquor and antibiotics.

9. The method of claim 1 wherein said inoculation mixture is incubated at least seven hours.

10. The method of claim 1 wherein a second quantity of an antibacterial agent is added during said seeding.

11. The method of claim 1 which further includes the step of transferring said inoculation mixture to a seeding tank prior to said seeding.

12. The method of claim 1 wherein said seeding mixture is incubated for at least seven hours.

13. The method of claim 1 which further includes the step of transferring said seeding mixture to a propagation tank prior to said propagating.

14. The method of claim 1 wherein a third quantity of an antibacterial agent is added during said propagating.

15. The method of claim 1 wherein a fourth quantity of an antibacterial agent is added during said combining.

16. The method of claim 1 which further includes the step of transferring said propagating mixture to a fermentation tank prior to said fermenting.

17. The method of claim 1 which further includes the step of extracting a quantity of protein feed.

18. The method of claim 1 which further includes the step of checking the ratio of solids to liquids of said propagation mixture.

19. The method of claim 1 which further includes the step of checking the ratio of solids to liquids in said fermentation mixture.

20. The method of claim 1 which further includes the step of checking the volumetric percentage ratio of alcohol in said fermentation mixture prior to said extracting said quantity of ethanol and said quantity of yeast.

21. The method of claim 1 which further includes the step of maintaining the temperature of said incubation of said inoculation mixture at 85° F.

22. The method of claim 1 which further includes the step of maintaining the temperature of said incubation of said seeding mixture at 88° F.

23. The method of dam 1 which further includes the step of maintaining the temperature of said propagation mixture between 90° F. and 91° F.

24. The method of claim 1 which further includes the step of maintaining the temperature of said propagating mixture during said fermentation between 95° F. and 98° F.
